SEQUENCE LISTING (1 sequence)

SEQ ID NO: 1 - DNA, 7046 bp, Mus musculus
cgcctgagttgcccgcgacggctctgcccgccgacggcacgtctctcggcggccgcgcgttccggggaagttacgtgcgggagcaggctttggaggagacgcccgagggtgtaggggacagccggaggcccgggtactgcggagcggcgagccggcggagggcggcggaggccgagcaggcggccgggtgtgccccgcggagaagcccgggcggggcggacgcttcccggacttttgtcccacttgaatcccctccccgtgggccgggcctttccggcctcccccgcccctgccccgctcgtccccgggagatgtttatgcggacggtggcgtgaggagcgggcgggccggcggcgcggagtttcgggtccgaggagcttcgcgcggcgcggagagagacaagatgtccgccagggccgcggccgctaagagcacagcaatggaggaaacagctatatgggaacagcacacagtgacgcttcacagggctcctgggtttggatttggaattgcaatctctggtggaagagataatcctcattttcagagtggggaaacctccatagtgatttctgatgtgttaaaaggagggccagctgaaggacagctacaggaaaatgaccgagttgcaatggttaacggagtttcaatggataacgttgaacatgcttttgctgttcagcagctaaggaagagtgggaaaaacgcaaaaattactatccgaaggaagaagaaagttcagatccctgtaagtcacccagatcctgagccggtgtctgataatgaagacgatagttatgacgaagaagtgcatgacccaagagctggccgcggtgctttagcgaacagaaggagcgagaagagctgggcaagggataggagtgcaagcagggagaggagcctgtcccctcgctcggacaggcggtccgtggcctccagtcagcccgcaaagcccaccaaggtcacactggtgaagtctcggaaaaatgaagaatatggtcttcgaccggccagccacatatttgtaaaggaaatttcacaagatagtttggcagcaagagatggtgacattcaagaaggggatgttgtcttgaagataaatggtactgtgacagaaaatatgtcattgacagatgcaaaaacactgatagaaaggtctaaaggcaagttaaaaatggtagtgcaaagagatgagcgggctaccttactgaacgtccctgacctttcggatagtatccattctgctaatgcctcggaaagagatgacatttcagaaattcagtcactagcgtcagaccattcaggtcgctcgcatgacaggccaccccgccgcagccagtcacgatctcctgaccaacgttcagagccctccgatcattccacgcagtctccacagcagcccagcaatggcagtctccggagcagagaggaagagcgaatgtctaaacctggggccatctcaactcctgtaaaacatgtagacgatcatccacccaaagcagtggaagaagttacagttgagaaaaatgagaagcagacgcccactcttccagaaccgaaacctgtgtatgctcaagttggacaaccagatgtggatttacccgtcagcccttctgatggtgctctgcctaattcagctcatgaagacgggatacttaggcccagcatgaaactggtaaaattcagaaaaggagatagtgtgggtttgcgactagctggtggaaatgatgtcggaatatttgtagctggcgttctagaagatagccctgcagccaaagaaggcttagaggaaggtgatcaaattctcagggtgaacaatgtagatttcacaaatatcataagggaagaggccgtccttttcctccttgacctccctaaaggtgaagaagtgaccatactggctcagaagaagaaggacgtttatcgccgcattgtagaatcagatgtaggagattcattctatattagaacgcattttgaatatgaaaaagaatctccttacggacttagttttaacaaaggagaggtgttccgggtcgtggatactttatacaatggaaagctgggctcttggcttgccattcgaattggcaaaaatcataaggaggtagaacgaggcatcatcccaaataagaacagagctgaacagttagccagtgtacagtacacacttccaaagacagcgggtggtgatcgggcagacttctggaggtttcgaggtcttcgcagctccaagagaaatcttcgaaaaagcagagaggacttgtcagctcagccagttcaaactaagttcccagcttatgaaagggttgttcttcgagaagctggattcctaagacctgtaaccatctttggaccaatagctgatgttgccagagaaaagttggcaagagaggagccagatatctatcagattgcaaaaagtgaactacgagacgctgggactgaccatcgtagctctggcatcattcgccttcatacaataaagcaaatcatagatcaggataaacatgctttattagatgtaacgccaaatgcggttgatcgtcttaattatgcgcagtggtatccaattgttgtgttccttaaccctgactctaagcaaggtgtaaaaacaatgaggatgaggttgtgtccggagtctcggaaaagcgccaggaagctatatgaacgctctcataagcttcgtaagaacaatcaccatctcttcacaactacaattaacttaaactcaatgaatgatggttggtacggtgccctgaaagaagcgattcagcagcaacagaaccagctggtgtgggtctctgaggggaaggcggatggtgctacaagtgatgaccttgatttgcatgacgatcgtctgtcctacctgtcagccccaggtagtgagtactcaatgtatagcacggacagtagacacacttctgactatgaagacacagatacagaaggcggggcctacactgatcaagaactagatgaaactcttaatgatgaggtggggactcccccggagtctgccattacacggtcctctgagcctgtaagagaggattcctctggaatgcatcatgaaaaccagacataccctccttactcaccacaagcgcagccacaagctattcatagaatagactcccctggacttaagccagcctctcaacagaaagcagaagcctcatctccagtcccttacctttcgcctgaaacaaccccagcatcatcagcctctgcagttaatcataatgtcagtgtaactaatgtcagcctggaggagcctgccccagcccctcccacctcgcacgcatcacagcctggttgtttaggagcaccaagtgctgaggcagctcacgtaggtctcagaggtgaaggaccaccattgccgccgcatgcagacccagcaaaggtgtacaggaaggagccatattctgaagaaatgatgagacaaaaccatattttaaaacaaccagctcttggtcacccagggcagaggccagataaagagccaaatctagcctatgaaccccaacttccatatatagaaaaacaagccagcagagaccttgagcagccgtcatacaggtatgaggtctcaagctacacagaccagttttctcggaactatgaccatcgcctacggtttgaagatcgaatccctacctatgaagaccagtggtcatattatgatgacaaacagccctaccaacctcggccttttgagaatcagcatccccgagacctggactccagacaacatcccgaagaggcttcagaacgaggttatttccagcgttttgaagagccagcccctctgtcgtacgacagtagaacacgctatgagcagctgcctcgaacctctactctacgacatgaagagcagccagcccctgcatatgaggtgcacaacaggtacaggccagaggcacagccctattcttcaacaggccctaagtcatctgagcccaagcagtactttgaccagtacccgcgaagttatgagcaagttccaccaccaggctttacctccaaaacaggccattacgagcctctccatggtgctgcagttgtccctcctctgataccttcctctcaacaaaagccagaagtcctgcgctcggctaccaaaccacagcctccacccccaaccctaactgaggaggaggaggatccagcaatgaaaccacagtctgtgctcaccagagtcaaaatgtttgaaaacaaaagatctgcgtctttggagaacaagaaagatgtgaatgacactgccagcttcaagcctccggaagtagcatctaaacctccaggtgcttctcttgctggccctaaacctgtccctcagagtcagtttagtgagcacgacaaaacgctctacaggctcccagagcctcagaaacctcaagtgaagccacccgaagatattgttcgatcaaatcattacgaccctgaagaggatgaagaatattaccggaaacagctctcctactttgaccgaagaagttttgagagcaagccttctgcacatcttcctgctggccatcactcagagcctgctaagccagtccattctcagagccagcccaacttccctagttactcttcaaagggaaaacccgaaactgatgctgtggatagatcattcagtgagaaacgttatgatccagcccaggccacgcctcctcctcctccgttgccctcacagtacagccagccagctccacctctgtccagctcttctctccacatacattccaagggcgcccagggtgaaggcaactcagtatcattggattttcagaactcatatatgtccaaaccagacccacccccatctcagagtaaaccagcaactttcagaccaccaactcgagaagacccccctcagactttctatccgcagaaaagtttcccagacaaagctccagttaacggagctgagcagactcagaaaaccatcactccggtgtacaatcgattcacaccaaagccgtacacaagttctgcccggccatttgaacgcaaatttgaaagtccgaagttcaaccataatcttctgccaagtgaaactgtacataaacctgaattgtcttcaaaaactcccacttccccaaaaactcttatgaaagctcatagttcaacacagcctccagagtttggcagtggagttgaaactttctctgttcacacagataagcctaaatatcaaatgaataatatcagcaccatgcctaaagctgtccctgtgagtccttcagctgtggaagaagatgaagatgaggatggtcatactgtagtggctacagctcgtggcatttttaacagcaatggtggtgtgttgagttccatagaaaccggtgttagtataattatcccacaaggagccattcctgaaggaattgagcaagaaatctatttcaaagtctgcagagacaatagcattctcccacctttagataaagagaaaggtgaaactctgctgagccccctagtgatgtgtgggccccatggcctcaagttcctgaagcccgtggagctacgcttgccacactgtgaccctaaaacctggcaaaacaagtgtcttcctggagatccgaattaccttgttggagccaactgtgtttctgtcctgattgaccacttttaattcttagtgtataggaactggactaagcaatgtgaacgtggattgaacttactaaatctaaatggaaccactctaatgagtattatattttcttagaatttatactgcaattggtagtattaagcatttgttggaactgatgaaggttagcgagcatgcccctgagccacggtcagaaagcatgctacaagctatgtgttattgagtgaagaactgtcaggcattggctagaggttcaaagatatttttgctttgtaatgatttttgtacttttttatggtcactgcttaacttcacatactgatttccgttaaaaataccagccagtaaatgggggtgcatttgagttctgttctttccaaagtacactcaaagtttattatggccttggcctagcatacacattttattttattatacatgaggtaatgtgcacacattttttacaaatgcacctggaatatataaccagtatagtggatttaacagaaatgtacagcagggggatttataacttggggagggagggtcaaatgaagacaattacttattgtatatgaaaacacatttcttttagggaaggacaccaaagcatgtgagcccggttcatggcctctgaatctataaattaacatatcaccacagacatgtaaccagcaggaatgccttaccctagtgggtttaattcttccatcatttcgctgtgtattactaagtttttatgagttctgtgcatatctagatactgtaccatggaaaagactgagtagattgtggacttgatggtttcagaaatgtgcaaattacctggaagaaaataatggtgtgacttaaaatatttttttgacagggggagtggcagtcttctgttctttgggctgtgcatctgagatgtttgatgctttcaggacgcatgtacataatgcgtgcataccacttttgttcttggtttgtaaattaacttttataaactttacctttttatacataaacagaaccaagtttctaaggctacctttgtattctctcctgtacctcttgagccttgaactttgacctctgcagcaataaagcagcatttctacacatacaaggtcatttttctaagaaaaagaatgcacagagttgttacatttttaagtgctgcatttaaaagatacagttactctagtttgattaaattcttgcaaagtatcccttctgtaaaatttgtgatacaatactgtgccctaaagtgtatttttttactaatagacagtttattatggcacatcagcacgatttctgtttagataatacaccactacattctgttaataatcagtaggtgtgaaaggatttcttttgtggttattaaaaaaaaatctcaaatttctaaatctgcaagaataaaactttttaaaat

(19) United States
(12) Patent Application Publication
(10) Pub. No.: US 2006/0172419 A1
(43) Pub. Date: Aug. 3, 2006

(54) Highly efficient gene targeting vectors and methods for gene targeting method forward epithelial cell line

(75) Inventors: Kazuaki Umeda, Otsu-shi (JP); Takeshi Matsui, Kyoto-shi (JP); Mayumi Nakayama, Kyoto-shi (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Eisai Co., Ltd., Tokyo (JP)

(21) Appl. No.: 10/539,534

(22) PCT Filed: Dec. 24, 2003
(86) PCT No.: PCT/JP03/16549

§ 371 Date: Feb. 13, 2006

(30) Foreign Application Priority Data
Dec. 24, 2002 (JP) 2002-371621
Mar. 20, 2003 (JP) 2003-76877

(51) Int. Cl. A01K 67/027; C12N 5/06; C12N 15/87

(52) U.S. Cl. 435/455; 435/354; 800/18

(57) ABSTRACT

In the process of generating ZO-1 gene knockout mice, the present inventors developed gene targeting vectors that allow homologous recombination to occur at a high probability (90% or more). In addition, the present inventors used the targeting vectors for studying optimal electroporation conditions in epithelial cells, and as a result, successfully determined optimal electroporation conditions that would enable efficient gene targeting in the EpH4 mouse epithelial cell line. Use of the vectors of the present invention enables easy introduction of exogenous genes into the ZO-1 allele of ES cells. Moreover, since effects on the genomic structure need not be considered, the methods of the present invention are expected to solve problems encountered in the conventional methods for generating transgenic mice and stable transformants.

### TECHNICAL FIELD

The present invention relates to vectors capable of high-efficiency gene targeting. The present invention also relates to methods for efficient gene targeting in epithelial cell lines, particularly the EpH4 mouse epithelial cell line.

### BACKGROUND ART

Gene targeting (Non-patent Document 1: A. L. Joyner, “Gene Targeting”, Second Edition, Oxford University Press) is a strategy of gene disruption or gene transfer which introduces an exogenous DNA fragment into a mammalian cell and causes homologous recombination with an endogenous DNA sequence. This method is widely used, particularly in mouse embryonic stem cells (ES cells), for generating various mutations in many genes and for expressing exogenous genes. Mice deleted in a target gene or mice expressing an exogenous gene can be generated by transferring ES cells back into mice. Physiological functions of the target gene can be deduced by analyzing the phenotypes of these mice at an individual level. However, frequency of homologous recombination is generally very low, i.e. only 0.1 to 1% in cells that have been introduced with the exogenous gene. If this frequency increases, free alteration of genes would become possible, and applications can be expected in various fields of medicine and research. However, vectors that are capable of efficient gene targeting are yet unknown.

The term “epithelial cells” refers to monolayered or multilayered cell sheets covering body surfaces, lumen surfaces, and such. Upon establishing a definite cell polarity within the cells themselves, epithelial cells become specialized and carry out the function of separating each organ into a closed space with a distinct environment. 90% or more of human malignant tumors are known to derive from epithelial cells. Therefore, it is also medically important to analyze the mechanisms of epithelial cell formation and their maintenance functions. Since epithelial cells are formed at the early stages of development, detailed analysis of genes important for the formation and function of epithelial cells in knockout mice often fails.

While primary gene targeting methods (see Non-patent Reference 1, supra) used so far in mouse ES cells utilize homologous recombination, it is known that the efficiency of homologous recombination in differentiated somatic cells is very low and ranges from 0.001 to 0.01% (see, non-patent reference 2: Sedivy J M and Dutriaux A, “Gene targeting and somatic cell genetics-a rebirth or a coming of age?”, Trends Genet. 15:88-90 (1999)).

Thus, gene targeting in epithelial cells is very difficult, and methods for efficient gene targeting in epithelial cells are still unknown.

### DISCLOSURE OF THE INVENTION

The present invention was achieved in view of the above circumstances. An objective of the present invention is to provide highly efficient gene targeting vectors. Another objective is to provide methods for efficient gene targeting in epithelial cells, particularly the EpH4 mouse epithelial cell line.

The present inventors performed extensive studies to solve the above issues. In the process of generating a knockout mouse lacking the ZO-1 gene which encodes a scaffolding protein localized in the tight junctions of epithelial cells, the present inventors developed gene targeting vectors that enable homologous recombination with a 90% or higher probability. In addition, they investigated the optimal electroporation conditions for using the vectors in epithelial cells, and determined optimal electroporation conditions that would allow efficient gene targeting in the EpH4 mouse epithelial cell line, and thereby completed the present invention.

Exogenous genes can be readily introduced into ZO-1 alleles in ES cells by utilizing the vectors of the present invention. Since no phenotypes of any sort was observed in single knockout ES cells obtained using the targeting vectors of the present invention, or in hetero mice generated by transferring the single knockout ES cells into mice, the introduction of an exogenous gene into one of the ZO-1 alleles is considered to have no effect on cellular functions. This is advantageous when expressing an exogenous gene because there is no need to consider the effects on genome structure. In other words, the methods of the present invention are expected to overcome drawbacks in the conventional methods of generating transgenic mice and producing stable transformants.

The efficiency of gene targeting in somatic cells has always been considered to be very low (0.001 to 0.01% in cells introduced with an exogenous gene). However, by optimizing gene transfer according to the present invention, it was shown that even in the EpH4 epithelial cell line, approximately 10% of the cells introduced with an exogenous gene undergo homologous recombination.

It is expected that functions of genes expressed in epithelial cells can be efficiently analyzed by using the gene targeting methods of the present invention. Moreover, since these methods can be used to disrupt genes associated with malignant tumor formation as well as to introduce genetic variants into a genome by knock-in methods, the present invention enables the use of epithelial cells with altered genes in the development of drug screening systems.

Until now, gene targeting via homologous recombination has been successful only in a few cell lines. However, by using the vectors of the present invention to study optimal conditions for gene targeting, the application of gene targeting to the generation of gene disrupted cell lines suitable for drug screening and pathological analysis is also expected.

As described above, the present inventors discovered that when generating animals introduced with exogenous genes, high-efficiency gene targeting could be achieved by making the ZO-1 gene a target site for introducing exogenous genes. That is, the inventors of the present invention discovered for the first time that vectors using the ZO-1 gene as a target site allow introduction of exogenous genes at a high efficiency. Moreover, the inventors discovered methods which use these vectors for gene targeting in epithelial cells, particularly in the EpH4 mouse epithelial cell line, and thereby completed the present invention.

The present invention relates to highly-efficienct gene targeting vectors and methods that utilize these vectors for gene targeting in epithelial cells, particularly the EpH4 mouse epithelial cell line. More specifically, the present invention provides:

(1) a gene targeting vector for introducing an exogenous gene into the region of the ZO-1 gene of a non-human animal, wherein the vector comprises said exogenous gene and an entire or a partial region of said ZO-1 gene;

(2) the vector of (1), wherein the vector comprises a structure in which the entire or partial region of the ZO-1 gene has been placed upstream and/or downstream of the exogenous gene;

(3) the vector of (2), wherein the partial region of the ZO-1 gene comprises exon II or a part thereof;

(4) the vector of (3), wherein the vector comprises a structure in which either DNA fragments (a) or (b) indicated below are placed at either side of the exogenous gene, respectively:
(a) a 1.5 kb Bsp1286I-Bsp1286I fragment comprising a part of exon II and upstream thereof within the ZO-1 gene, and a 8.5 kb PstI-BamHI fragment located downstream of exon II;
(b) a 5.1 kb PstI-BsrDI fragment comprising a part of exon II and upstream thereof within the ZO-1. gene, and a 3.9 kb PstI-SphI fragment located downstream of exon II;

(5) a gene targeting vector for introducing an exogenous gene into the region of the ZO-2 gene or the Disabled-2 gene of a non-human animal, wherein the vector comprises said exogenous gene and an entire or a partial region of said ZO-2 gene or said Disabled-2 gene;

(6) the vector of (5), wherein the vector comprises a structure in which the entire or a partial region of the ZO-2 gene or the Disabled-2 gene has been placed upstream and/or downstream of the exogenous gene;

(7) the vector of any one of (1) to (6), wherein the vector is used for generation of a non-human animal expressing the exogenous gene or a non-human animal cell expressing the exogenous gene;

(8) the vector of any one of (1) to (7), wherein the vector comprises a promoter capable of transcribing the exogenous gene has been provided upstream thereof;

(9) the vector of any one of (1) to (8), wherein the vector further comprises a marker gene expression cassette;

(10) the vector of (9), wherein the vector comprises a structure in which the exogenous gene has been placed adjacent to downstream of the marker gene expression cassette;

(11) the vector of any one of (1) to (6), wherein the exogenous gene is the marker gene expression cassette;

(12) the vector of any one of (9) to (11), wherein the marker gene expression cassette is a drug resistance gene expression cassette;

(13) the vector of (12), wherein the drug resistance gene expression cassette is a DNA fragment comprising β-geo;

(14) the vector of any one of (1) to (13), wherein the non-human animal is mouse;

(15) a method for gene targeting in a epithelial cell, wherein the method comprises introducing a targeting vector into said cell by electroporation under the conditions of voltage at 0.4 to 0.5 kV and capacity of condenser at 125 to 250 μF;

(16) the method of (15), wherein calcium concentration in the cell preparation solution used for electroporation is 5 μM or less;

(17) the method of (15) or (16), wherein the targeting vector targets the ZO-1 gene, the ZO-2 gene or the Disabled-2 gene on the chromosome of the cell;

(18) the method of (15) or (16), wherein the targeting vector is any one of (1) to (13);

(19) the method of any one of (15) to (18), wherein the epithelial cell is derived from a higher animal cell;

(20) the method of (19), wherein the higher animal is mouse;

(21) the method of (20), wherein the cell is EpH4 mouse epithelial cell line;

(22) a method of producing a epithelial cell line in which the chromosome has been artificially altered, wherein the method comprises introducing the targeting vector into the epithelial cell by the gene targeting method of any one of (15) to (21).

The present invention relates to gene targeting vectors which allow efficient introduction of exogenous genes into chromosomes.

The present invention provides targeting vectors comprising a target site for insertion of an exogenous gene, wherein the target site is the ZO-1 gene region, the ZO-2 gene region, or the Disabled-2 gene region in non-human animals.

The present invention provides gene targeting vectors (simply referred to as “vectors” in some cases) for introducing exogenous genes into the ZO-1 gene region, the ZO-2 gene region or the Disabled-2 gene region in non-human animals. The vectors of the present invention are useful for generating non-human animals or cells that express exogenous genes.

Nucleotide sequences of the ZO-1 gene are already known, and information related to ZO-1 gene nucleotide sequences can be easily obtained from a public gene bank known to those skilled in the art. For example, information related to the nucleotide sequence of the mouse ZO-1 gene can be obtained from GenBank using the Accession No: NM_{—}009386. Information related to the ZO-2 gene nucleotide sequence and the Disabled-2 gene nucleotide sequence are also available from GenBank (Accession No: NM_{—}011597 and Accession No: NM_{—}023118, respectively). The nucleotide sequence of the mouse ZO-1 gene obtained from GenBank (Accession No: NM_{—}009686) is shown in SEQ ID NO: 1 as an example of a ZO-1 gene nucleotide sequence.

The vectors of the present invention are targeting vectors that comprise an exogenous gene and an entire or partial region of the ZO-1 gene, ZO-2 gene or Disabled-2 gene. It is possible to generate transgenic animals comprising chromosome-integrated exogenous genes by utilizing the vectors of the present invention.

The term “gene targeting” refers to the artificial alteration of a chromosomal gene by utilizing the homologous recombination phenomenon that takes place between DNA molecules having the same nucleotide sequences. Here “alteration” includes gene disruption and gene insertion. That is, by introducing into a cell a vector having a sequence homologous to a chromosomal target sequence, the vector (or a portion thereof) is inserted into the chromosome through recombination between homologous portions.

In one preferable embodiment of the present invention, the vector has a structure comprising, on both sides of an exogenous gene, DNA fragments consisting of nucleotide sequences homologous to the DNA regions on both sides of a chromosome site (target site) for insertion of the exogenous gene. When the above-described vector of the present invention is introduced into cells, homologous recombination occurs between the homologous DNA regions at both sides of the exogenous gene and the target site, and the exogenous gene is inserted into the chromosomal target site.

In the present invention, the potential chromosomal DNA site that may serve as a target for exogenous gene insertion is not limited, as long as it is located on the ZO-1 gene, the ZO-2 gene, or the Disabled-2 gene, and may also include gene expression regulatory regions such as exons, introns, and promoters. Thus, in one preferable embodiment of the present invention, the vector has a structure in which an entire or partial region of the ZO-1 gene, the ZO-2 gene, or the Disabled-2 gene is placed upstream and/or downstream of the exogenous gene.

In addition, the exogenous gene may be inserted into any region as long as it is on the ZO-1 gene, ZO-2 gene, or Disabled-2 gene.

For example, when the ZO-1 gene is selected as a target site, the exogenous gene is preferably inserted into a DNA region containing exon II of the ZO-1 gene or a portion thereof. Accordingly, the ZO-1 gene region comprised in the vector of the present invention is preferably, for example, a DNA region of the ZO-1 gene comprising an entire or portion of exon II. The DNA region comprising a portion of the above exon II includes specific examples such as (a) a 1.5 kb Bsp1286I-Bsp1286I fragment containing a portion of exon II and its upstream region, and an 8.5 kb PstI-BamHI fragment located downstream of exon II, or (b) a 5.1 kb PstI-BsrDI fragment containing a portion of exon II and its upstream region, and a 3.9 kb PstI-SphI fragment located downstream of exon II, but is not limited thereto.

Those having a structure in which the two fragments mentioned above are respectively placed upstream and downstream of the exogenous gene can exemplify the vectors of the present invention.

The present invention also provides methods that allow efficient gene targeting in epithelial cells. Various methods for introducing a vector into cells are known. An example of the most popular methods is the electroporation method. Those skilled in the art can perform electroporation appropriately using generally sold equipments and materials.

The methods of the present invention are gene-targeting methods comprising the step of introducing a targeting vector into epithelial cells by electroporation.

According to the methods of the present invention, electroporation may be carried out using the Gene Pulser II System (Bio-Rad Laboratories) or the Pulse Controller PLUS, under the voltage and condenser capacity conditions of preferably 0.4 to 0.5 kV and 125 to 250 μF, and more preferably 0.45 kV or around 0.45 kV and 125 μF or around 125 μF. These electroporation conditions which allow highly efficient gene targeting in epithelial cells were determined for the first time by the present inventors.

In addition, cells (culture solutions) used in electroporation may also be suitably prepared by those skilled in the art using various known methods. For example, cells used in electroporation may be prepared by adding calcium. The inventors of the present invention discovered that when cells with strong intercellular adhesion are used in electroporation, lowering the calcium concentration in cell preparations increases gene transfer efficiency (see Example 2 below). Thus, the final calcium concentration in the cell culture at the time of cell preparation is preferably 5 μM or less, but is not limited thereto.

The targeting vectors of the present invention used in the above methods are preferably, for example, the above-described vectors. Specifically, in one preferable embodiment of the present invention, the vector is constructed to target the ZO-1 gene (GenBank Accession No: NM_{—}009386), the ZO-2 gene (GenBank Accession No: NM_{—}011597), or the Disabled-2 gene (GenBank Accession No: NM_{—}023118) on an epithelial cell chromosome, but it is limited thereto, and can also be constructed to target any DNA region on the epithelial cell chromosome.

In addition, the targeting vectors of the present invention used in the above methods may have an entire or partial region of the target gene on the epithelial cell chromosome. The targeting vectors of the present invention used in the above methods may include, for example, those having an above-described exogenous gene and an entire or partial region of the ZO-1 gene (referred to as “ZO-1 vector” in some cases).

Moreover, one example of the vectors of the present invention is a targeting vector (ZO-2 vector) that targets the ZO-2 gene. The ZO-2 vector is a vector having an exogenous gene and/or an entire or partial region of the ZO-2 gene. More specifically, the ZO-2 vector is a vector that comprises a region of the ZO-2 gene, where exon III of the ZO-2 gene is deleted. The vector may be constructed using, for example, a 6.6 kb Kpn I-Hind III fragment located upstream of exon III and a 2.6 kb Sca I-Spe I fragment located downstream of exon III.

In addition, the vectors of the present invention include, for example, a targeting vector (Disabled-2 vector) that targets the Disabled-2 gene. The Disabled-2 vector is a vector having, for example, an exogenous gene and/or an entire or partial region of the Disabled-2 gene. More specifically, the Disabled-2 vector is a vector that comprises a region of the Disabled-2 gene, where exon III of the Disabled-2 gene is deleted. For construction of the Disabled-2 vector, two 3.9 kb fragments located upstream and downstream of exon III may be obtained by genomic PCR, using genomic DNA of the EpH4 epithelial cell line as template. Here, an EcoRV site may be introduced at the 3′ side of the upstream fragment to allow Southern blot analysis utilizing the newly introduced site.

The above-mentioned exogenous genes of the present invention are generally protein-encoding genes that can be used to express proteins, but are not limited thereto. The proteins expressed from an above-mentioned vector include examples of desired proteins, which can be natural or artificial proteins. Natural proteins include, for example, secretory proteins, membrane proteins, cytosolic proteins, nuclear proteins, hormones, cytokines, growth factors, receptors, enzymes, and peptides. Artificial proteins include, for example, fusion proteins such as chimeric toxins, dominant negative proteins (including soluble molecules of receptors or membrane-bound dominant negative receptors), deletion-type cell adhesive molecules, and cell surface molecules. Moreover, they may be proteins with added secretion signals, membrane localization signals or nuclear localization signals. It is also possible to suppress the expression of a gene whose expression is not desired, or to suppress the function of its gene product, by expressing antisense RNA molecules, RNA-cleaving ribozymes, or such.

An above-mentioned exogenous gene of the present invention preferably has the so called “expression cassette” structure, in which the gene is operably linked to a promoter so as to be transcribed. The above phrase “operably linked” means that a promoter is linked to a downstream gene so that the expression of the downstream gene is induced upon activation of the promoter.

In addition to the above-mentioned promoter which includes one originally comprised in the gene itself, promoters known to those skilled in the art are available and can be randomly selected by taking into consideration the exogenous gene type and the type of cells into which the exogenous gene is introduced. Specifically, the promoters of the present invention include examples such as cytomegalovirus (CMV)-derived promoter, EF-1α promoter, β actin promoter, Rous sarcoma virus (RSV)-derived promoter, SV40 promoter, TK promoter, PGK promoter, SRα promoter, but are not limited thereto.

There are no limitations on the chain length of the DNA homologous to a target gene region (simply referred to as “homologous DNA” in some cases) comprised in an above-mentioned vector, as long as it allows homologous recombination. Since it is generally known that the longer the length of a homologous region, the higher the efficiency of homologous recombination, the DNA region homologous to a targeted gene region comprised in a vector of the present invention is also preferably long. Generally, for homologous recombination in mammalian cells such as mouse ES cells, the preferable total length of the “homologous DNAs” located on both sides of the exogenous gene is 5 kb or more, and preferably at least 0.5 to 2 kb on each side. Accordingly, the “homologous DNAs” comprised in a vector may generally have a total length of 5 kb or more. The “homologous DNAs” located on both sides of the exogenous gene are preferably 0.5 kb or more, and more preferably 2 kb or more on each side.

Moreover, to select cells that have an exogenous gene introduced into their chromosomes, and to confirm introduction of the exogenous gene, the vectors of the present invention preferably comprise a marker gene expression cassette. The term “marker gene expression cassette” refers to a cassette constructed to express a marker gene, which is a DNA fragment having a general structure in which the marker gene is operably linked to one or a number of promoters, internal ribosome entry sites (IRESs), and splicing accepters (SAs).

For example, a drug resistance gene such as neomycin resistance gene (neo) may be used for the above mentioned “marker gene”. When a drug resistance gene is inserted into a vector, a cell line that has undergone homologous recombination may be selected through cell culturing in a medium containing the drug. Specifically, a marker gene such as β-geo (in the order of lacZ, neo, and polyA), neomycin resistance gene, hygromycin resistance gene, or puromycin resistance gene may be suitably used.

In addition, when an above-mentioned vector comprises an above-mentioned marker gene expression cassette, the vector preferably has a structure in which an exogenous gene is placed adjacent to the downstream (3′ side) of the expression cassette, but the structure is not limited thereto. Furthermore, an “exogenous gene” of the present invention may be a marker gene expression cassette mentioned above.

Moreover, the targeting vectors of the present invention also include a vector having an entire or partial region of a target gene without having an exogenous gene. For example, gene targeting vectors having an entire or partial region of the ZO-1 gene without having an exogenous gene are also included in the present invention. That is, when an above-mentioned vector is provided, an exogenous gene and a marker gene expression cassette can be cloned into appropriate sites of the vector by those skilled in the art using standard means. For easy insertion of an exogenous gene and if necessary a marker gene expression cassette, into the above vector, cloning sites may be designed as the insertion sites. Such cloning sites may be restriction enzyme recognition sites. This allows insertion of an exogenous gene and a marker gene expression cassette as necessary into the restriction sites. The cloning site includes the so-called multi-cloning site having a sequence recognized by a multiple restriction enzyme. These cloning sites are designed and constructed using genetic engineering techniques commonly known to those skilled in the art.

Moreover, when introducing a vector into a cell, to select cells that carry the vector, an above vector may comprise several kinds of marker genes different from the above marker genes, as necessary.

There are no limitations on the basic structure of the targeting vectors used in the present invention, and commercially available vectors including, for example, pBluescript, pGEM vector (Promega Corporation), and pUC vector (Takara Bio Inc.), may be suitably used. The targeting vector may be constructed utilizing, for example, a commercially available vector using genetic engineering techniques commonly known to those skilled in the art.

Animals whose chromosomes can be introduced with an exogenous gene utilizing the vectors of the present invention are preferably, for example, mice, but are not limited thereto, as long as they are non-human animals carrying the ZO-1 gene or a homolog thereof. The dog. ZO-1 gene is known as a homolog of the mouse ZO-1 gene, which suggests that in principle the ZO-1 gene is expressed in all mammals. Therefore, the vectors of the present invention may be useful for gene targeting in all mammals.

As an example, cells of higher animals can be suitably used in the gene targeting methods of the present invention. Higher animal cells include those from, for example, mice, pets and domestic animals, and those of experimental animals such as dogs, rats, hamsters, rabbits, pigs, bovines, horses, chickens, monkeys, sheep, goats, and cats.

Specific examples of cells used in the gene targeting methods of the present invention include the EpH4 mouse epithelial cell line (Reichmann, E., Ball, R., Groner, B., and Friis, R. R., “New mammary epithelial and fibroblastic cell clones in coculture form structures competent to differentiate functionally”, J. Cell Biol. 108:1127-1138 (1989)), MTD-1A cell line (Enami, J., S. Enami, and M. Koga, “Isolation of an insulin-responsive preadipose cell line and a mammary tumor-producing”, dome-forming epithelial cell line from a mouse mammary tumor, Dev. Growth Differ. 26:223-234 (1984)), CSG211 cell line (Wigley C B and Franks L M., “Salivary epithelial cells in primary culture: Characterization of their growth and functional properties”, J. Cell Sci. 20:149-165 (1976)). Among them, the EpH4 mouse epithelial cell line is most preferable. Moreover, cells derived from these cell lines may also be suitably used.

In one preferable embodiment of the present invention, generally, a targeting vector of the present invention is introduced into epithelial cells by electroporation under the conditions described above, and then the cells are cultured. Cells carrying the exogenous gene inserted into their chromosomes through homologous recombination are then selected by using an appropriate selection marker.

The gene targeting methods of the present invention may be used, for example, to generate cells into which an exogenous gene has been introduced.

The cells which comprise and express an introduced exogenous gene can be easily generated using the vectors of the present invention by methods known to those skilled in the art.

In addition, non-human transgenic animals such as transgenic mice may be generated using the vectors of the present invention described below without being limited thereto. Non-human transgenic animals other than mice can also be generated by general genetic engineering techniques using the vectors of the present invention. First of all, a vector of the present invention comprising an exogenous gene is introduced into a mouse ES cell line by electroporation, and then cells that have undergone homologous recombination are selected. A chimeric mouse can be obtained by injecting the thus obtained cells into mouse blastocysts. The chimeric mice are mated to generate a mouse in which the exogenous gene is introduced into one of the ZO-1 alleles on the mouse chromosome. The mouse thus obtained may be mated further to generate a mouse in which the exogenous gene is introduced into both alleles of the ZO-1 gene on the mouse chromosome.

Expression levels of an exogenous gene inserted into the chromosome by methods using the vectors of the present invention or the above methods of the present invention may be assayed using methods known to those skilled in the art. Transcriptional gene products may be detected by, for example, Northern hybridization, RT-PCR, RNA protection assay, and such. Detection of transcripts by Northern hybridization, RT-PCR, and such may also be performed in situ. In addition, Western blot analysis, immunoprecipitation, RIA, ELISA, pull down assay, and such, which use antibodies, may be used to detect translational gene products. Moreover, for easy detection of gene transfer, a vector that produces a protein of interest with a tag, or one that expresses a reporter gene in addition to an exogenous gene, may also be constructed. The reporter genes include genes encoding β-galactosidase, chloramphenicol acetyltransferase (CAT), alkaline phosphatase, GFP (Green Fluorescent Protein), or such, without being limited thereto. An appropriate reporter gene may be selected depending on the cell type by one skilled in the art.

The present invention provides methods for producing epithelial cell lines having artificially altered chromosomes, comprising introducing a targeting vector into an epithelial cell line by a gene targeting method of the present invention. The targeting vectors introduced by the methods of the present invention are generally inserted into chromosomes as a whole or in part through homologous recombination between the vector and the epithelial cell chromosome. The above-mentioned “epithelial cell lines having altered chromosomes” include epithelial cell lines in which an exogenous gene is inserted into the chromosome, epithelial cell lines comprising an altered endogenous gene, and such. The alteration includes, for example, insertion, addition, deletion, and substitution of DNA.

### BRIEF DESCRIPTION OF THE DRAWINGS

FIG. 1 is a schematic representation of the mouse ZO-1 gene locus and the gene targeting vector. The initiation codon is located within exon I.

The targeting vector comprising β-geo (lacZ/neo/polyA) at the center of the ZO-1 gene is made by deleting a portion of exon II. The 3′ probe used for Southern blotting is shown as a black line.

FIG. 2 is a photograph showing the results of ZO-1 single knockout cell generation. Southern blotting of the ZO-1 gene locus digested with PvuII using the 3′ probe yielded a 6.3 kb band from the wild type gene locus and a 4.7 kb band from the gene locus after gene disruption (all lanes except for lanes 13, 15, and 17). Lanes 13, 15, and 17 are from clones that were not targeted. Among the 176 G418-resistant colonies, 167 colonies underwent single homologous recombination.

FIG. 3 shows gene targeting of the ZO-1 gene in the EpH4 mouse epithelial cell line: mouse ZO-1 gene locus, targeting vector, and restriction enzyme map of the mouse ZO-1 gene locus after gene disruption.

The initiation codon is located within exon I. The targeting vector is made to contain a neomycin cassette (lacZ/β-geo/polyA) in the center of the ZO-1 gene so that a portion of exon II is removed. The 5′ probe used for Southern blotting is shown as a black line.

FIG. 4 shows gene targeting of the ZO-2 gene in the EpH4 mouse epithelial cell line: mouse ZO-2 gene locus, targeting vector, and restriction enzyme map of the mouse ZO-2 gene locus after gene disruption. The targeting vector is made to contain a neomycin cassette (IRES/β-geo/polyA) in the center of the ZO-2 gene so that exon III is removed. The 3 ′ probe used for Southern blotting is shown as a black line.

FIG. 5 is a photograph showing the results of Southern blot analysis using the probe shown in FIG. 4.

FIG. 6 shows gene targeting of the Dsabled-2 gene in the EpH4 mouse epithelial cell line: mouse Disabled-2 gene locus, targeting vector, and restriction enzyme map of the mouse Disabled-2 gene locus after gene disruption. The targeting vector is made to contain a neomycin cassette (IRES/β-geo/polyA) in the center of the Disabled-2 gene so that exon III is removed. The 5′ probe is shown as a black line.

FIG. 7 is a photograph showing the results of Southern blot analysis using the probe shown in FIG. 6.

FIG. 8 is a graph showing effects of the voltage and condenser capacity on the number of G418-resistant colonies.

Gene transfer of the ZO-1 gene targeting vector was carried out at different voltages and condenser capacities, and then colonies formed were selected by G418 and counted. The results show that the number of colonies increases as the voltage and condenser capacity increase.

FIG. 9 is a photograph of electrophoresis showing results of the Southern blot analysis for confirming gene transfer in the ZO-1 single knockout EpH4 cells.

Southern blot analysis of the EcoRI-digested ZO-1 gene locus using the 5′ probe detected a 20.9 kb band from the wild type gene locus and a 5.3 kb band from the gene locus after gene disruption (lanes 1, 2, 11, and 12). Among the 142 G418-resistant colonies, 16 colonies underwent single homologous recombination.

### BEST MODE FOR CARRYING OUT THE INVENTION

The present invention will be specifically described below with reference to Examples, but it is not to be construed as being limited thereto.

### EXAMPLE 1

### Construction of Various Highly Efficient Targeting Vectors

(1) Construction of the ZO-1 targeting vector

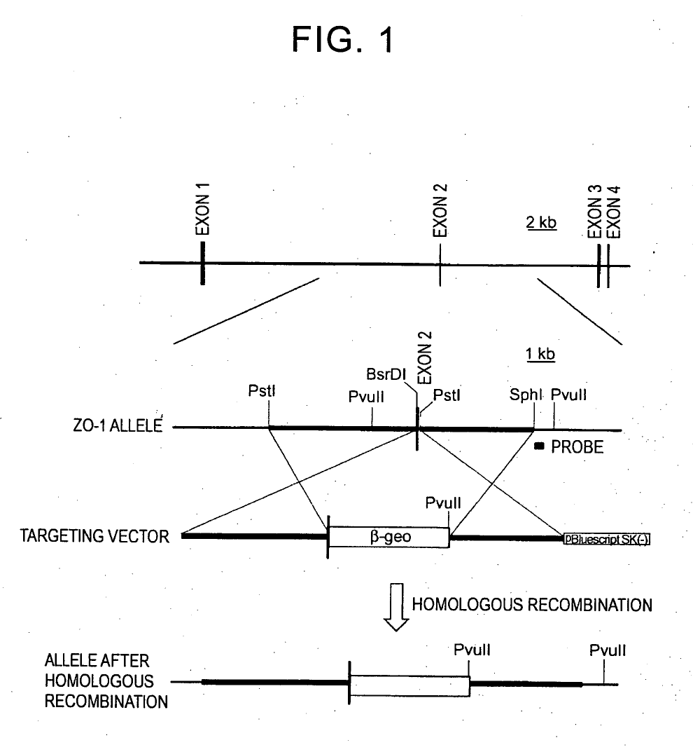

To obtain a genome comprising ZO-1, 129/Sv mouse genomic λ phage library was screened using a mouse ZO-1 cDNA probe, and four overlapping genomic fragments were obtained. Mouse ZO-1 genomic locus was determined by restriction enzyme mapping and DNA sequencing (FIG. 1). A genomic fragment comprising the ZO-1 gene was obtained. The genomic fragment consisted of four exons. The initiation codon was located in exon I.

The ZO-1 targeting vector was designed so that a portion of exon II is removed. This vector contains β-geo (in the order of lacZ, neo, and polyA) as a cassette. A 5.1 kb PstI-BsrDI fragment containing a portion of exon II and its upstream was ligated to the upstream of the above cassette, and a 3.9 kb PstI-SphI fragment located downstream of exon II was ligated to the downstream the above cassette (FIG. 1). With the use of this targeting vector, a region containing the exon II portion is removed and β-geo is inserted instead when homologous recombination occurs between the targeting vector and the ZO-1 gene.

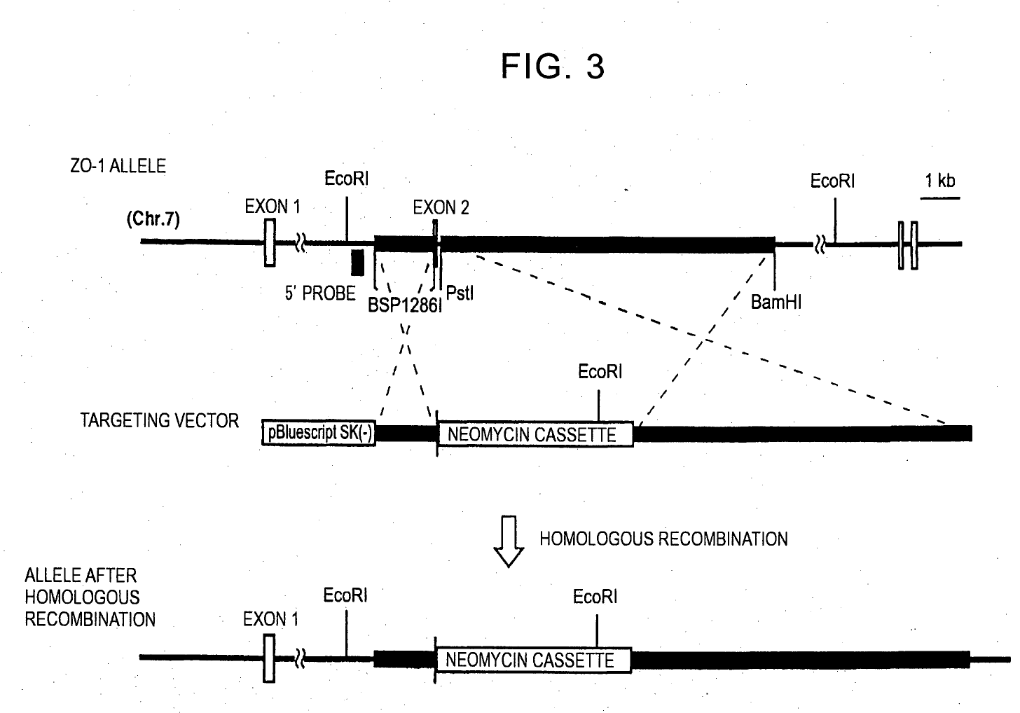

Alternatively, the above targeting vector may comprise a neomycin cassette (in the order of lacZ, neo, and polyA) as a cassette. In such a case, a 1.5 kb Bsp1286I-Bsp1286I fragment containing a portion of exon II and its upstream, and an 8.5 kb PstI-BamHI fragment located upstream of exon II, were respectively ligated to the upstream and downstream of the above cassette (FIG. 3).

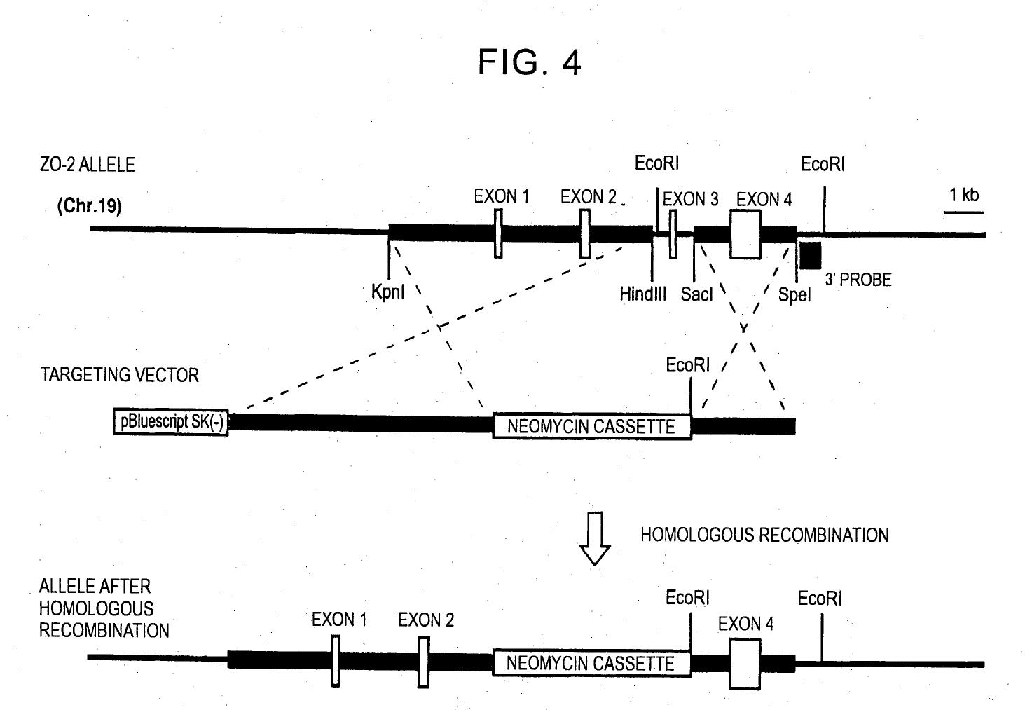

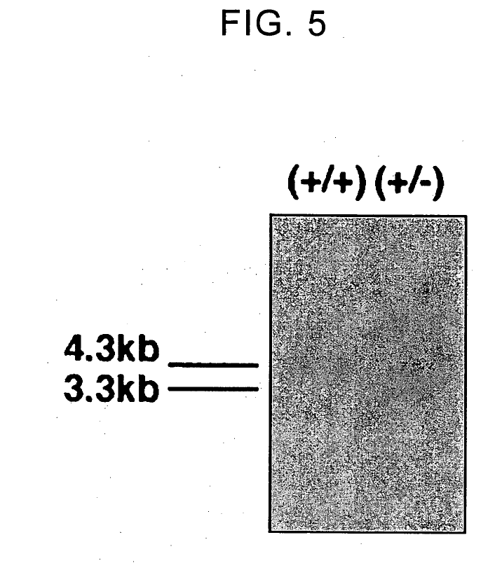

(2) The ZO-2 targeting vector was designed so that exon III was removed. This vector includes a neomycin cassette (in the order of loxP, En-2, SA [splicing accepter], IRES [internal ribosome entry site], β-geo, polyA, and loxP) as a cassette. In addition, En-2 which is a sequence containing engrailed-2 gene intron and splicing accepter is thought to enhance splicing efficiency. A 6.6 kb KpnI-HindIII fragment located upstream of exon III and a 2.6 kb ScaI-SpeI fragment located downstream of exon III were ligated respectively to the upstream and downstream of the above vector (FIGS. 4 and 5). With the use of this targeting vector, exon III-containing region is removed when and homologous recombination occurs between the targeting vector and the ZO-2 gene.

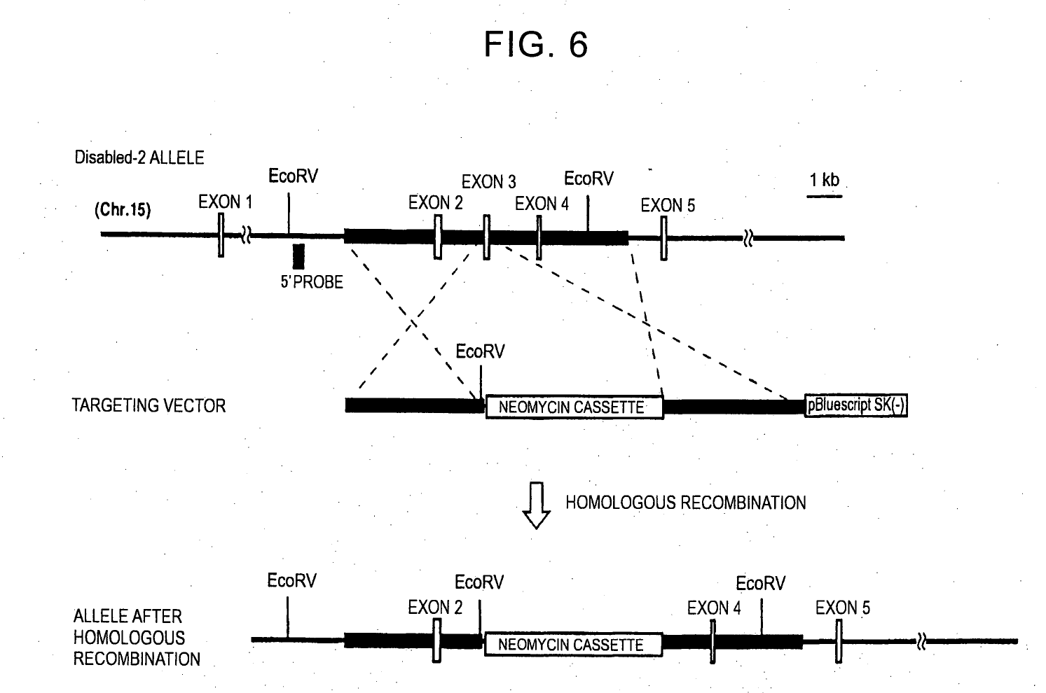

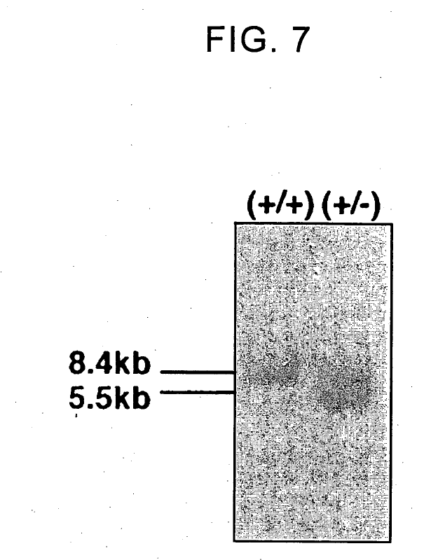

(3) The Disabled-2 targeting vector was designed in such a way that exon III is removed. This vector contains a neomycin cassette (in the order of loxp, SA, IRES, β-geo, loxp, and polyA), similarly to the ZO-2 targeting vector. Two 3.9 kb fragments located upstream and downstream of exon III, respectively, were obtained by genomic PCR using genomic DNA of the EpH4 epithelial cell line as template, and ligated respectively to the upstream and downstream of the above cassette (FIGS. 6 and 7). At the same time, an EcoRV site was introduced at the 3′ end of the upstream fragment. This newly introduced site was used for Southern blot analysis. With the use of this targeting vector, the exon III-containing region is removed when homologous recombination takes place between the targeting vector and the Disabled-2 gene.

### EXAMPLE 2

### Gene Targeting

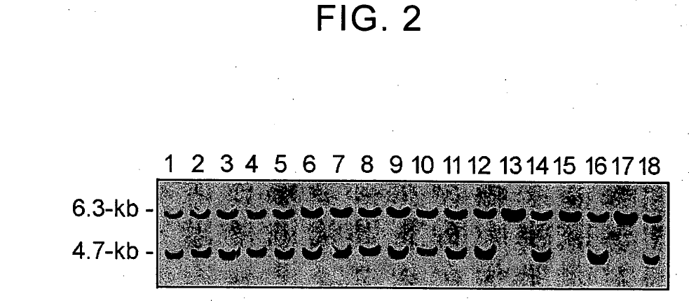

The ZO-1 targeting vector was linearized at the unique SacII site located at the 5′ end of the 5′ homologous region fragment. The vector thus linearized was used for gene transfer into ES cells (7-8 passages) by electroporation using the Gene Pulser (Bio-Rad Laboratories). These ES cells were cultured on feeder cells in a standard medium for 36-48 hours, and then cultured for an additional 7-13 days in a medium containing G418 (175 mg/ml). G418 resistant colonies were isolated and screening was carried out by Southern blotting using a genomic fragment (290 bp) that corresponds to the outside of the 3′ homologous region fragment. In clones that have undergone correct homologous recombination, when their genome is digested with PvuII, a 6.3 kb fragment could be seen from the wild type allele and a 4.7 kb fragment could be seen from the allele replaced by homologous recombination (FIG. 2). Among the 176 G418-resistant clones, 167 clones were found to have undergone homologous recombination. That is, a targeting efficiency of approximately 95% was confirmed. The results indicate that this vector is more efficient for homologous recombination compared with conventional vectors.

In addition, the inventors of the present invention confirmed that efficient gene targeting of the ZO-1 gene via homologous recombination can also be applied to another cell line, F9 teratocarcinoma cells.

### EXAMPLE 3

### Investigation of Electroporation Conditions for Gene Targeting

In order to develop gene targeting methods for the EpH4 mouse epithelial cell line, conditions for electroporation were examined using the efficient ZO-1 targeting vector (targeting vector comprising the 1.5 kb and 8.5 kb fragments) described in Example 1.

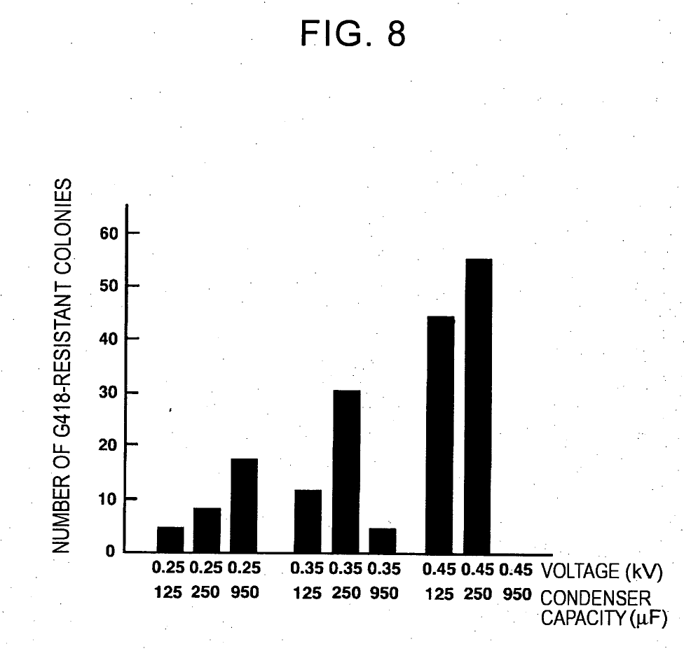

The mouse ZO-1 gene targeting vector was linearized at the unique XhoI site located at the 3′ end of the 3′ homologous region fragment (FIG. 3). This DNA (10 μg) was introduced into the EpH4 mouse epithelial cell line (1×10⁷ cells) cultured in a low Ca²⁺ (5 μM) medium for 12 hours, by electroporation using the Gene Pulser II system (Pulse Controller PLUS) from BioRad Laboratories under different conditions of voltage, condenser capacity, and such. Following the introduction, cells were cultured in a standard medium for 48 hours and selected after 14 days of culturing in a G418-containing medium (0.4 mg/ml). As a result, differences in the number of G418 resistant colonies due to different electroporation conditions were observed (FIG. 8). This indicates that the gene transfer efficiency of the targeting vector varies depending on the electroporation condition.

Subsequently, G418 resistant colonies isolated under the different electroporation conditions were grown and each cell's genome extracted. Southern blot analysis was performed using a genomic fragment (280 bp) corresponding to the outside of the 5′ homologous region fragment. As a result, the most efficient conditions for homologous recombination were found to be 0.45 kV and 125 μF (Table 1).

TABLE 1

| | Number of hits/ | |
|---|---|---|
| Voltage, Condenser | total number of | Targeting efficiency |
| capacity | clones analyzed | (%) |
| 0.25 kV, 125 F | 0/6 | 0 |
| 0.25 kV, 250 F | 0/9 | 0 |
| 0.25 kV, 950 F | 1/18 | 5.5 |
| 0.35 kV, 125 F | 1/12 | 8.3 |
| 0.35 kV, 250 F | 1/31 | 3.2 |
| 0.35 kV, 950 F | 0/6 | 0 |
| 0.45 kV, 125 F | 4/46 | 8.6 |
| 0.45 kV, 250 F | 3/55 | 5.4 |
| 0.45 kV, 950 F | 0/0 | 0 |

The optimal conditions for electroporation thus determined were different from those obtained using ES cells.

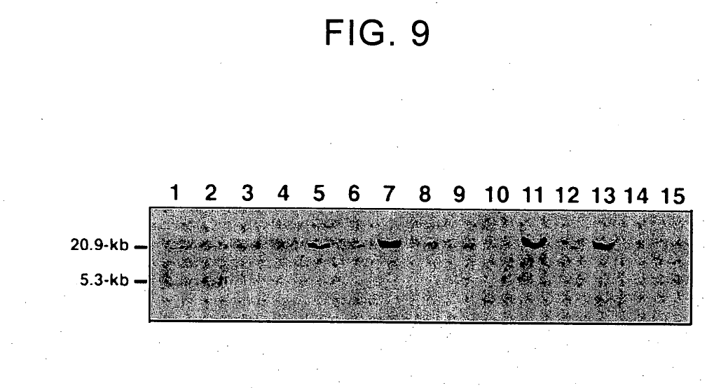

When gene targeting was carried out again for the ZO-1 gene locus under the conditions thus determined, 16 clones (11.2%) out of the 142 clones were found to have undergone homologous recombination. When the genomic DNA from clones that have undergone correct homologous recombination at the ZO-1 gene locus was digested with EcoRI, a new 5.3 kb band was detected in addition to the 20.9 kb band from the wild type allele (FIG. 9).

### Industrial Applicability

Use of the vectors of the present invention enables easy introduction of exogenous genes into the ZO-1 allele in ES cells. Since no phenotypes of any sort were observed in the single knockout ES cells obtained using the targeting vectors of the present invention or in the hetero mice generated by transferring the single knockout ES cells into mice, introduction of exogenous genes into one of the ZO-1 alleles does not affect cellular functions. This is advantageous when expressing exogenous genes in that effects on genomic structure need not be considered. In other words, this approach is expected to solve the underlying problems of conventional methods for constructing transgenic mice and stable transformants.

In addition, functions of genes expressed in epithelial cells may be analyzed using the gene targeting methods of the present invention. The methods of the present invention allow disruption of genes associated with malignant tumor formation, as well as efficient knock-in of the genetic variants into genomes. Thus it may also be possible to develop drug screening systems using epithelial cells with altered genes.

1. A gene targeting vector for introducing an exogenous gene into the ZO-1 gene region in a non-human animal, wherein the vector comprises the exogenous gene and an entire or partial region of the ZO-1 gene.

2. The vector of claim 1, comprising a structure in which the entire or partial region of the ZO-1 gene is placed upstream and/or downstream of the exogenous gene.

3. The vector of claim 2, wherein the partial region of the ZO-1 gene comprises exon II or a portion thereof.

4. The vector of claim 3, comprising a structure in which either one of the DNA fragments in (a) and (b) below is placed at each side of the exogenous gene:
(a) a 1.5 kb Bsp1286I-Bsp1286I fragment comprising a partial exon II of the ZO-1 gene and its upstream, and an 8.5 kb PstI-BamHI fragment located downstream of exon II;
(b) a 5.1 kb PstI-BsrDI fragment comprising a partial exon II of the ZO-1 gene and its upstream, and a 3.9 kb PstI-SphI fragment located downstream of exon II.

5. A gene targeting vector for introducing an exogenous gene into the ZO-2 gene region or the Disabled-2 gene region of a non-human animal, wherein the vector comprises the exogenous gene and an entire or partial region of the ZO-2 gene or the Disabled-2 gene.

6. The vector of claim 5, wherein the vector comprises a structure in which an entire or partial region of the ZO-2 gene or the Disabled-2 gene is placed upstream and/or downstream of the exogenous gene.

7. The vector of claim 1 or 5, wherein the vector is used for generating a non-human animal expressing an exogenous gene or a non-human animal cell expressing an exogenous gene.

8. The vector of claim 1 or 5, wherein the vector comprises in the upstream of an exogenous gene a promoter capable of transcribing the exogenous gene.

9. The vector of claim 1 or 5, wherein the vector further comprises a marker gene expression cassette.

10. The vector of claim 9, wherein the vector comprises a structure in which an exogenous gene is placed adjacent to the downstream of a marker gene expression cassette.

11. The vector of claim 1 or 5, wherein the exogenous gene is a marker gene expression cassette.

12. The vector of claim 9, wherein the marker gene expression cassette is a drug resistance gene expression cassette.

13. The vector of claim 12, wherein the drug resistance gene expression cassette is a DNA fragment comprising β-geo.

14. The vector of claim 1 or 5, wherein the non-human animal is a mouse.

15. A method for epithelial cell gene targeting, wherein the method comprises introducing a targeting vector into the cell by electroporation under the conditions of 0.4 to 0.5 kV voltage and 125 to 250 μF condenser capacity.

16. The method of claim 15, wherein calcium concentration in a prepared cell solution used for electroporation is 5 μM or less.

17. The method of claim 15 or 16, wherein the targeting vector targets the ZO-1 gene, the ZO-2 gene, or the Disabled-2 gene on cellular chromosome.

18. The method of claim 15, wherein the targeting vector is the targeting vector of claims 1 or 5.

19. The method of claim 15, wherein the epithelial cell is derived from a higher animal cell.

20. The method of claim 19, wherein the higher animal is a mouse.

21. The method of claim 20, wherein the cell is the EpH4 mouse epithelial cell line.

22. A method for producing an epithelial cell line having an artificially altered chromosome, wherein the method comprises introducing a targeting vector into an epithelial cell line by the gene targeting method of claim 15.

23. The vector of claim 11, wherein the marker gene expression cassette is a drug resistance gene expression cassette.

24. The vector of claim 23, wherein the drug resistance gene expression cassette is a DNA fragment comprising β-geo.
